(19) United States
(12) Patent Application Publication
(10) Pub. No.: US 2026/0091196 A1
(43) Pub. Date: Apr. 2, 2026

(54) PROVISIONING DEVICE FOR STORING AND TRANSPORTING CATHETERS

(71) Applicant: B. Braun Melsungen AG, Melsungen (DE)

(72) Inventors: Eberhard Engelien, Wohltorf (DE); Fred Held, Hamburg (DE); Verena Desch, Hamburg (DE); David Rieche, Hamburg (DE)

(21) Appl. No.: 19/369,754

(22) Filed: Oct. 27, 2025

Related U.S. Application Data
(63) Continuation of application No. PCT/EP2024/061587, filed on Apr. 26, 2024.

(30) Foreign Application Priority Data
Apr. 28, 2023 (DE) 10 2023 111 146.9

(51) Int. Cl. A61M 25/00; A61M 25/02; A61M 25/09; A61M 25/10

(52) U.S. Cl. CPC A61M 25/002; A61M 25/10; A61M 2025/024; A61M 25/09; A61M 2209/06

(57) ABSTRACT

A provisioning apparatus can be used for the storage and transport of catheters, for example balloon catheters for percutaneous transluminal coronary angioplasty. The provisioning apparatus includes a support and a holding device arranged or formed on the support for holding a catheter rolled up in an annular manner in a storage position. The holding device includes at least three holding elements that are in engagement with the catheter in the storage position. The at least three holding elements are arranged or formed spatially separate from one another on the support.

### CROSS-REFERENCE TO RELATED APPLICATIONS

This application is a continuation of International Application No. PCT/EP2024/061587, filed on Apr. 26, 2024, and claims priority to German Application No. 10 2023 111 146.9, filed on Apr. 28, 2023. The contents of International Application No. PCT/EP2024/061587 and German Application No. 10 2023 111 146.9 are incorporated by reference herein in their entireties.

### FIELD

The present disclosure relates to a provisioning apparatus for the storage and transport of catheters, in particular of balloon catheters for percutaneous transluminal coronary angioplasty.

### BACKGROUND

Balloon catheters for percutaneous transluminal coronary angioplasty, also referred to as PTCA catheters, serve to treat high-grade coronary stenoses. To treat them, the balloon catheter is inserted via a guide wire into a coronary artery and is inflated.

PTCA catheters that are currently available on the market are transported and stored in so-called dispensers. This is a long plastic hose that is longer than the catheter itself. The plastic hose is rolled with the catheter accommodated therein to form a circle and is fixed against unrolling by means of clips. Inserted catheters can be securely transported and stored in this type of packaging.

Problems of the described dispensers are, for one, their handling, in particular the cumbersome removal of the catheter and, for another, their environmental impact. Just the plastic hose used as packaging constitutes the largest share of the plastic requirement of the entire product, namely about 95%.

A holding apparatus, in particular for medical equipment, is disclosed in JP 2016-064089 A. JP 2012-176101 A relates to a packaging for medical devices, in particular catheters. A storage apparatus for objects having a long extent is described in WO 2019/171672 A1. DE 43 33 073 A1 discloses a packaging for hose-shaped goods. U.S. Pat. No. 4,332,322 discloses a folding packaging for holding a spool of a plastic tube, said packaging having clips and corresponding molded pieces. U.S. Pat. No. 5,131,537 relates to a packaging for medical catheters. US 2005/0194276 A1 describes a packaging for elongate medical devices and methods for producing and using the same.

### SUMMARY

It is therefore an object of the present disclosure to improve a provisioning apparatus of the kind described at the outset.

This object is achieved, in accordance with the present disclosure, in a provisioning apparatus of the kind described at the outset in that the provisioning apparatus comprises a support and a holding device arranged or formed on the support for holding a catheter rolled up in an annular manner in a storage position, in that the holding device comprises at least three holding elements that are in engagement with the catheter in the storage position, and in that the at least three holding elements are arranged or formed spatially separate from one another on the support, in that the at least three holding elements are positioned along a holding circle, which defines a holding circle midpoint, in that at least one, in particular all, of the at least three holding elements are configured in the form of a holding tab, in that a free holding tab end of the holding tab is oriented pointing in the direction toward a support center of the support, and in that the holding device comprises at least two retaining elements for preventing a movement of the catheter in the storage position in the direction toward the holding circle midpoint.

A provisioning apparatus further developed as proposed makes it possible, in particular, to hold a rolled up catheter on the support in a simple manner. The at least three holding elements serve this purpose. In particular, only three holding elements may be provided. They serve to support the rolled up catheter, for example, at three points or at three short contact portions. Such an arrangement makes it possible, in particular, to handle the catheter in a simple manner. In particular, both ends of the catheter can thus be held freely accessible on the provisioning apparatus or on the support thereof. This significantly improves a handling of the provisioning apparatus compared to the known packaging apparatus described at the outset. In particular, depending on the concrete arrangement of the holding elements, it is possible to remove the catheter as a whole, namely in its rolled up state, from the provisioning apparatus. This is not possible in the case of a catheter stored in a plastic hose. In the case of such a hose storage, the catheter must first be pulled completely out of the hose in order to make both ends freely accessible at the same time. The provisioning apparatus further developed as proposed in particular also has a significantly better environmental balance. In particular, a support can be configured very flat, such that, for one, material for producing the same can be save compared to a plastic hose and, for another, the provisioning apparatus with a catheter held thereon, which is also able to cling to the support as flat as possible, is able to be configured even flatter than the described known hose packaging. Space requirements for the product can be significantly reduced in this way. Production, transport, and storage costs can thus be significantly reduced. Positioning the at least three holding elements along a holding circle, which defines a holding circle midpoint, makes it possible in a simple manner to hold a catheter rolled up in the shape of a circular ring on the support. In particular, the holding circle midpoint can be positioned in the region of a support center by correspondingly arranging the at least three holding elements. It is advantageous that at least one, in particular all, of the at least three holding elements is/are configured in the form of a holding tab and that a free holding tab end of the holding tab is oriented pointing in the direction toward a support center of the support. In particular, the support center may be the holding circle midpoint. Holding tabs can be formed on the support in a simple manner, in particle by adhesion or alternatively by being integrally formed from the support itself. This is described in more detail below. Provision is further made that the holding device comprises at least two retaining elements for preventing a movement of the catheter in the storage position in the direction toward the holding circle midpoint. In particular, the retaining elements can, as a supplement to the holding elements, further improve the storage of the catheter on the provisioning apparatus. The proposed provisioning apparatus is advantageous in particular for PTCA catheters. Said catheter may comprise a wire that forms a sort of restoring device in the form of a resilient element in order to stabilize the catheter in an elongated form. This is advantageous, in particular, upon insertion of the catheter into blood vessels of a patient. However, the wire may also cause the catheter to snap out of a rolled up position into its elongated basic position if the catheter is not secured in a suitable manner. In particular, the proposed provisioning apparatus remedies this, because by means of the at least three holding elements in engagement with the catheter in the storage position, of which at least one prevents a movement of the catheter in the storage position in the direction toward the holding circle midpoint and at least one prevents a movement away from the holding circle midpoint, it positions the catheter in a secure and defined manner and is able to hold the catheter in the rolled up storage position on the support. Such a mounting is not necessary for so-called OTW catheters, which are flexible. OTW catheters comprise no stabilizing wire, such that such a catheter is also not able to simply snap into an elongated basic position, as is the case with PTCA catheters.

It is favorable if the provisioning apparatus is configured to bring the rolled up catheter and the at least three holding elements out of engagement without bringing the catheter into an elongated form. As already explained, it is thus possible to remove the catheter as a whole, i.e., in a rolled up position, from the provisioning apparatus. Both ends of the catheter are then freely accessible.

Advantageously, two of the at least three holding elements are positioned point-symmetrically relative to the holding circle midpoint. If a third holding element is positioned between the two holding elements along the holding circle, this results in a free region extending over nearly 180° between the holding elements located opposite one another relative to the holding circle midpoint. This makes it possible, in particular, to hold the catheter securely on the provisioning apparatus, but also to remove it in a rolled up state from the provisioning apparatus. To do this, it is only necessary to slightly deform the rolled up catheter.

It is favorable if the at least three holding elements define a holding region, which correspond to a holding circle circumferential angle of the holding circle of at least 180°. In this way, it is possible to hold a rolled up catheter securely on the provisioning apparatus.

It is favorable if the holding circle circumferential angle is in a range between 180° and 240°. In particular, it may be in a range of about 185° to about 200°. For a reliable mounting, it is advantageous if the holding circle circumferential angle is greater than 180°.

In accordance with a further preferred embodiment, provision may be made that the support has an outer support rim, which encloses a storage region, and that the catheter in the storage position is arranged within the storage region. This configuration makes it possible, in particular, to hold the catheter on the provisioning apparatus in a protected manner. Even if a user bumps the provisioning apparatus with the catheter stored thereon with the support rim against an object, the catheter is still well protected against damage.

It is advantageous if the holding tab is formed by a holding cut line formed on the support and having two free ends, if the holding tab defines a holding tab base line, which connects the two free ends of the holding cut line, and if a free holding tab length of the holding tab is defined by a distance of the free holding tab end from the holding tab base line. To form the holding tabs, it is only necessary to form a cut on the support in such a way that the holding cut line has two free ends that are spaced at a distance from one another. Along the holding tab base line, the holding tab is then able to be pivoted out of a support surface defined by the support, in particular a support plane. It is also conceivable to bend or fold the holding tab along the holding tab base line. The catheter can be securely held on the support with holding tabs of that kind. Holding tabs that are bent out form, for example, wedge-shaped recesses in which the catheter can optionally even be held in a clamping manner.

For simple handling, it is favorable if the holding cut lines are convexly curved pointing in the direction toward the support center. Holding tabs having rounded holding tab ends can thus be formed, which keep the risk of damage to the catheter low.

The holding cut line is preferably of curved configuration at least in sections, in particular being configured in the form of a circular arc section. Holding cut lines of that kind can be formed in a simple manner by punch-cutting.

In order to form triangular or quadrangular holding tabs, it is favorable if the holding cut line comprises at least two, in particular three, holding line portions. These can be inclined relative to one another, for example.

In particular, it is advantageous if the at least two holding line portions are configured extending rectilinearly or substantially rectilinearly at least in sections. Triangular or quadrangular holding tabs can thus be created in a simple manner.

Preferably, the holding tab base lines of at least two holding tabs extend in parallel or substantially in parallel with one another. Such an arrangement is achieved, for example, if the two holding tabs are positioned point-symmetrically relative to the holding circle midpoint. They thus engage on opposite sides of the rolled up catheter in order to hold it on the provisioning apparatus.

It is advantageous if the holding tab base lines of at least two holding tabs extend transversely, in particular perpendicularly, to one another. Arranging two holding tabs in this way can ensure, in particular, that a rolled up catheter is not able to move in the direction of these two holding tabs to remove said catheter from the provisioning apparatus. In particular, a reliable securing of the catheter on the provisioning apparatus can thus be achieved.

In order to configure the support with minimal size, it is favorable if a distance of the holding tab base line of at least one holding tab from the support rim is smaller than its free holding tab length. A material usage for forming the provisioning apparatus can thus be minimized.

It is advantageous if at least one holding tab is positioned near the support rim and if its holding tab base line extends in parallel or substantially in parallel with the support rim. This design makes it possible, in particular, to minimize a size of the support.

It is favorable if at least one, in particular all, of the at least two retaining elements is/are configured in the form of a retaining tab and if a free retaining tab end of the retaining tab is oriented pointing in the radial direction away from the support center. In this way, in particular, a wedge-shaped receptacle pointing in the radial direction away from the support center can be formed by the retaining tab in order to accommodate a portion of the rolled up catheter. The retaining tab prevents, in particular, a movement of the rolled up catheter in the direction toward the support center or the holding circle midpoint.

It is advantageous if the retaining tab is formed by a retaining cut line formed on the support and having two free ends, if the retaining tab defines a retaining tab base line, which connects the two free ends of the retaining cut line, and if a free retaining tab length of the retaining tab is defined by a distance of the free retaining tab end from the retaining tab base line. In particular, as explained, retaining tabs can thus be formed integrally with the support. To achieve this, it is only necessary to form the retaining tab cut line, i.e., a corresponding cut, on the support. The retaining tab can then be brought into a desired position by deforming it relative to the support in order to, in particular, form a wedge-shaped receptacle for the rolled up catheter.

Favorably, the retaining cut lines are concavely curved pointing in the direction toward the support center at least in sections, in particular being configured in the form of a circular arc section. In this way, it is possible in a simple manner to create wedge-shaped receptacles that are open in the radial direction by means of the retaining tabs.

It is advantageous if at least one, in particular all, of the at least two retaining elements is/are positioned along the holding circle between the two holding elements that have the greatest angular distance from one another. In this way, the retaining elements do not significantly hinder a removal of the rolled up catheter from the provisioning apparatus.

Preferably, the retaining elements are positioned near the two stated holding elements, such that the rolled up catheter is optimally secured both in the direction toward the holding circle midpoint and away from it in the region where a retaining tab is arranged adjacent to a holding tab.

In accordance with a further preferred embodiment, provision may be made that the provisioning apparatus comprises an adapter holding device for a connection adapter arranged or formed on a first end of the catheter. This configuration makes it possible, in particular, to leave the catheter on the provisioning apparatus to prepare an intervention. It is not absolutely necessary to remove it. The catheter can be readily flushed by connecting the connection adapter, which is still held on the provisioning apparatus, namely on the adapter holding device. In particular, the catheter can hereby remain rolled up on the provisioning apparatus.

A connection adapter can be held on the provisioning apparatus in a secure manner if the adapter holding device comprises at least two adapter holding elements.

It is advantageous if two cooperating adapter holding elements are configured in the form of adapter holding tabs and if free adapter tab ends of the two adapter holding elements are oriented pointing toward one another. This makes it possible, in particular, to hold the connection adapter by means of the adapter holding tabs with the longitudinal axis of said connection adapter oriented in a direction transverse to the adapter tab ends pointing toward one another.

The adapter tab ends pointing toward one another are favorably spaced at a distance from one another on the support to form an adapter holding web. In this way, the connection adapter can be held stable on the provisioning apparatus, in particular supported by the adapter holding web.

It is advantageous if the adapter holding device defines an adapter holding longitudinal axis and if the adapter holding web extends in parallel or substantially in parallel with the adapter longitudinal axis. The connection adapter can thus be held, in particular, supported on the provisioning apparatus by the adapter holding web.

The provisioning apparatus can be formed in a simple manner if the adapter holding device is configured symmetrically relative to the adapter holding longitudinal axis.

The holding tab base line of a holding tab is advantageously oriented in parallel or substantially in parallel with the adapter holding longitudinal axis. A free end of the catheter, namely the first end with the connection adapter, can thus be guided, in particular in a kink-free manner, from the holding tab to the adapter holding device when being rolled up on the provisioning apparatus.

It is favorable if an adapter cutout is formed adjacent to the adapter holding device on the support in extension of the adapter holding longitudinal axis commencing from the support rim. Such an adapter cutout makes it possible, in particular, to position the connection adapter in such a way that a connection element thereof, which may be configured, e.g., in the form of a luer lock connector, can project from the provisioning apparatus. This makes it possible, in particular, to be able to connect the catheter, for example to a flushing apparatus for flushing the catheter, without having to remove the catheter, in particular the connection adapter, from the provisioning apparatus.

In accordance with a further preferred embodiment, provision may be made that the provisioning apparatus comprises a balloon holding device for an inflatable balloon arranged or formed in the region of a second end of the catheter. Such a balloon holding device enables, in particular, the secure positioning and mounting of the catheter in the region of the inflatable balloon.

It is favorable if the balloon holding device defines a balloon holding longitudinal axis and if the balloon holding device is configured asymmetrically relative to the balloon holding longitudinal axis. The balloon or a second end region of the catheter can thereby be reliably held on the provisioning apparatus.

The balloon holding device preferably comprises at least two balloon holding elements. For example, it may comprise two balloon holding elements, which may be configured like tabs.

It is advantageous if two cooperating balloon holding elements are configured in the form of balloon holding tabs and if free balloon holding tab ends of the balloon holding tabs, in an undeformed starting position of the provisioning apparatus, point in the same direction. This configuration makes it possible, for example, to reshape or fold over one of the two balloon holding tabs such that it supports the other balloon holding tab and is able to hold it in a position spread away from the support. The catheter can thus be positioned on the provisioning apparatus so as to be optimally protected in the region of the balloon.

It is favorable if each of the balloon holding tabs is formed by a balloon holding cut line formed on the support and having two free ends, if the balloon holding tab defines a balloon holding tab base line, which connects the two free ends of the balloon holding cut line, and if a free balloon holding tab length is defined by a distance of the free balloon holding tab end from the balloon holding tab base line. As already explained in the case of the holding tabs, balloon holding tabs for holding the catheter in the region of the inflatable balloon can thus be formed in a simple manner.

In order to keep the risk of damage to the catheter as low as possible, it is advantageous if the balloon holding cut lines are of curved configuration, in particular in the form of circular arc sections.

It is advantageous if the balloon holding tab base lines of the two cooperating balloon holding tabs extend in parallel or substantially in parallel with one another and with the balloon holding longitudinal axis. The catheter portion with the balloon can thereby be held in a rectilinear manner by the cooperating balloon holding tabs.

It is favorable if the balloon holding cut lines of the two cooperating balloon holding tabs differ in length and/or have different curvature radii. The balloon holding tabs can thus easily be configured having different sizes.

The balloon holding tab lengths of the two cooperating balloon holding tabs are advantageously equal or substantially equal. This can be achieved, in particular, even if the balloon holding cut lines differ in length or have different curvature radii. Balloon holding tabs having different sizes and different surface areas can thus be formed in a simple manner.

It is favorable if the holding tab base line of a holding tab is oriented in parallel or substantially in parallel with the balloon holding longitudinal axis. This configuration makes it possible, in particular, to spread the free second end of the catheter in the tangential direction away from the holding circle in a kink-free manner and to hold it with the balloon holding device.

In accordance with a further preferred embodiment, provision may be made that a tip recess is arranged or formed on the support and that a second end of the catheter in the storage position is positioned in the region of the tip recess. Such a tip recess easily enables a user to grasp a free tip, i.e., the second end of the catheter, in order to successively unwind the catheter from the provisioning apparatus starting from this end.

It is advantageous if the tip recess is positioned adjacent to the balloon holding device pointing in the direction of the balloon holding axis. This arrangement has the advantage, in particular, that the second end itself does not have to be supported, since it is reliably held on the provisioning apparatus adjacently in the region of the balloon.

It is favorable if the tip recess is configured in the form of a perforation on the support. Such a perforation can be formed in a simple manner, for example by punching. In particular, it may be configured in the shape of a circle.

For optimal handling of the provisioning apparatus, it is advantageous if the tip recess is dimensioned such that it makes it possible to grasp the second end of the catheter held on the provisioning apparatus with two fingers. For example, the free end can thus be easily grasped and handled with the thumb and index finger of one hand.

In accordance with a further preferred embodiment, provision may be made that a removal recess is arranged or formed on the support and that, in the storage position, at least two, in particular three, catheter portions of the catheter held rolled up on the support are positioned in the region of the removal recess. This configuration enables a user to securely grasp the catheter, namely two or more portions thereof at once, so that the risk of the catheter completely unrolling, i.e., transitioning into an elongated position, when removed from the support can be minimized. This makes it easier for a user to remove the catheter as a whole in its rolled up state from the provisioning apparatus.

The removal recess is favorably configured in the form of a perforation on the support. Such a perforation can be formed in a simple manner by punching. It enables free access to the rolled up catheter.

The removal recess is preferably configured in the form of a circular ring section. The removal recess may be configured, in particular, following a form of the rolled up catheter. In particular, this improves the handling of the provisioning apparatus and the catheter.

It is favorable if a center line of the circular ring section has a curvature that corresponds to about a curvature of the rolled up catheter held on the provisioning apparatus in the storage position. The catheter can thus be easily grasped by a user over an entire length of the removal recess in the circumferential direction relative to the holding circle midpoint and can be removed from the provisioning apparatus.

Preferably, the circular ring section extends over an angular range of about 30° to about 60°, in particular over an angular range of about 40° to about 50°.

It is advantageous if the removal recess has a width in the radial direction relative to the holding circle midpoint that makes it possible to grasp the catheter held on the provisioning apparatus with two fingers. A user can thus remove the catheter from the provisioning apparatus, in particular in a reliable manner and without damaging it.

The removal recess is preferably positioned on the holding circle between the two holding elements that have the greatest angular distance from one another. At the described position, the catheter can be grasped by a user and removed from the support, namely in particular in rolled up form, with the littlest exertion of force and without greater resistance by the holding tabs or the like.

In accordance with a further preferred embodiment, provision may be made that the provisioning apparatus comprises a wire holding device for holding a free end thereof, which projects from a wire channel of the catheter out of an opening of the wire channel pointing in the direction toward its first end. This configuration makes it possible, in particular, to hold the wire, which is typically used for transport purposes, on the provisioning apparatus in a targeted manner. In particular, it can thus be grasped by a user in a targeted manner. Preferably, the wire holding device is arranged or formed on the support in such a way that it is positioned neither on the holding circle nor on the adapter holding longitudinal axis. A free end of the wire can thus be held easily accessible separate from the rolled up catheter and also separate from the first end thereof. The wire holding device is favorably arranged or formed in a region between the holding circle and the adapter holding device.

The wire holding device favorably comprises at least one wire holding element. With said wire holding element, the wire is able to be reliably held on the provisioning apparatus in a desired orientation.

It is advantageous if the at least one wire holding element is configured in the form of a wire holding tab and if a free wire holding tab end of the wire holding tab is oriented pointing in the direction toward the support center. This configuration makes it possible, in particular, to reliably hold the free wire end on the provisioning apparatus. In particular, the orientation of the wire holding tab can prevent the free end from spreading away, in particular beyond a support rim of the support.

It is advantageous if the wire holding tab is formed by a wire holding cut line formed on the support and having two free ends, if the wire holding tab defines a wire holding tab base line, which connects the two free ends of the wire holding cut line, and if a free wire holding tab length of the wire holding tab is defined by a distance of the free wire holding tab end from the wire holding tab base line. As already explained in detail above in connection with the holding tab, such a wire holding tab can be formed integrally with the support in a simple manner.

The provisioning apparatus can be created in a simple and cost-effective manner if it is made of a flat material. A size, in particular a volume taken up by the provisioning apparatus, can thus also be kept minimal.

Preferably, the flat material forming a blank of the provisioning apparatus is a cardboard or paperboard sheet. The provisioning apparatus can be produced in a cost-effective and simple manner from said sheet, for example by punching. The flat material is favorably coated with a plastic material. In particular, the provisioning apparatus can thus be configured to be water resistant, namely even if the flat material is not water resistant, as is the case with cardboard or paperboard, for example.

It is favorable if the flat material forming a blank of the provisioning apparatus consists of plastic. This makes it possible, in particular, to form the support monolithically from a plastic material. In particular, such a provisioning apparatus can be configured to be water resistant. Furthermore, cut edges on a flat material made of a cardboard or paperboard sheet can cause problems, because such a sheet always contains fibers, the ends of which can then protrude freely. This can lead to fibers detaching from the support, which is undesirable in particular in a sterile environment. It may therefore be necessary to treat the cut edges of such a support made of a cardboard or paperboard sheet through an additional processing step, for example by coating or laminating the cut edges and optionally further regions of the support or by otherwise covering the partially exposed fibers or binding them to the support. This problem cannot occur in the case of a flat material made of plastic. The flat material may be configured, in particular, in the form of a film or in the form of a sheet.

In order to prevent as much as possible damage to a support made of a cardboard or paperboard sheet caused by liquids, it is advantageous if the support is provided with a liquid-repellent coating. For example, said coating may be configured to be water-repellent.

The liquid-repellent coating is preferably made of a plastic material. In particular, the coating may be formed by a plastic film with which the support is laminated.

Preferably, the plastic material for coating or forming the flat material is or contains polypropylene (PP), high-density polyethylene (HDPE), polylactic acid (PLA, in particular PLLA), polyamide (PA), polyethylene (PE), low-density polyethylene (LDPE), and/or polyethylene terephthalate (PET). Provisioning apparatuses can thus be formed in a simple and cost-effective manner.

The production of the provisioning apparatus can be further simplified if it is formed in one piece, in particular monolithically.

In order to keep a space requirement for the provisioning apparatus minimal, it is advantageous if the support has a support thickness in a range of about 0.05 mm to about 1 mm, in particular in a range of about 0.2 mm to about 0.4 mm.

In accordance with a further preferred embodiment, provision may be made that the provisioning apparatus has a front side and a rear side and that the holding device is configured to hold the catheter on the front side. In particular, all elements of the holding device and all other elements that have been described above can be arranged or formed on the provisioning apparatus in such a way that the catheter is able to be reliably fixed to one side of the support, namely on the front side, in the storage position.

It is favorable if the provisioning apparatus comprises at least one data storage element for storing characteristic data about the catheter, in particular length, diameter, inflation pressure, and balloon diameter. In particular, the data storage element may be an electronic data storage element. For example, it can be read wirelessly with corresponding reading devices. In particular, a documentation of medical procedures can thereby be simplified.

It is advantageous if the at least one data storage element is arranged on the front side or the rear side and is configured to be optically readable. For example, the data storage element may be configured in the form of a barcode or a QR-code. Optionally, the characteristic data about the catheter may also be printed in legible characters in a spatially delimited data region on the front and/or rear side, such that a user can themselves directly read all characteristic data for the catheter without having to use technical aids such as reading devices, for example.

In accordance with a further preferred embodiment, provision may be made that the provisioning apparatus comprises a catheter, in particular a PTCA catheter. Said catheter may comprise, in particular, a wire that forms a sort of restoring device in order to stabilize the catheter in an elongated form. This is advantageous, in particular, upon insertion of the catheter into blood vessels of a patient. The proposed provisioning apparatus is particularly advantageous in particular for this kind of catheter, because by means of the at least three holding elements in engagement with the catheter in the storage position, of which at least one prevents a movement of the catheter in the storage position in the direction toward the holding circle midpoint and at least one prevents a movement away from the holding circle midpoint, it positions the catheter in a secure and defined manner and is able to hold the catheter in the rolled up storage position on the support. For so-called OTW catheters, which are flexible, no mounting as proposed is necessary. OTW catheters comprise no wire, such that the catheter is not able to simply snap into an elongated basic position, as is the case with PTCA catheters.

It is favorable if the catheter has a first end and a second end, if a connection adapter is arranged or formed on the first end, and if an inflatable balloon is arranged or formed in the region of the second end. In particular, coronary stenoses can be optimally treated with such a catheter, as already explained in detail above, namely by dilating a coronary artery by means of the balloon.

In order to protect the catheter, in particular the balloon, it is advantageous if a removable protective cover surrounding the balloon is arranged on the second end.

In order to be able to inflate the balloon with a gas in a simple manner, it is advantageous if the catheter comprises a fluid channel, which fluidically connects the connection adapter to the balloon.

It is further favorable if the catheter comprises a wire channel for a wire and if the wire channel extends commencing from the second end in the proximal direction up to an insertion opening, which is arranged or formed on the catheter between the first and the second end. Preferably, it is arranged or formed closer to the second end than the first end where the connection adapter is arranged. The wire channel makes it possible, in particular, to guide the catheter to a coronary artery using a guide wire.

It is advantageous if the catheter comprises a wire arranged in the wire channel and if the wire is longer than the wire channel. The wire, which for example is positioned in the wire channel for transport purposes, thus projects out of the wire channel at one end, so that a user is able to easily grasp it and pull it out.

The wire is favorably configured in the form of a transport wire. It only serves the purpose of transport, in particular to hold the catheter on the provisioning apparatus as dimensionally stable as possible.

It is advantageous if the catheter is held on the provisioning apparatus in the storage position in such a way that the first end and/or the second end is/are oriented transversely, in particular perpendicularly, to the support rim. In particular, both the first end and the second end may be held on the provisioning apparatus pointing toward the same side of the support. In particular, this significantly improves the handling of the provisioning apparatus.

### BRIEF DESCRIPTION OF THE DRAWINGS

The subsequent description of a preferred embodiment of the present disclosure serves in conjunction with the drawings for further explanation.

FIG. 1 shows a schematic total view of a provisioning apparatus with a catheter held thereon in the storage position;

FIG. 2 shows a section view along line 2-2 in FIG. 1;

FIG. 3 shows an enlarged view of region A in FIG. 2;

FIG. 4 shows an enlarged view of region B in FIG. 2;

FIG. 5 shows a schematic view of the arrangement from FIG. 1 upon removal of the rolled up catheter from the provisioning apparatus;

FIG. 6 shows a view of the rolled up catheter;

FIG. 7 shows a view of a front side of the provisioning apparatus;

FIG. 8 shows a view of the rear side of the provisioning apparatus;

FIG. 9 shows a graphical explanation of a first option for removing the catheter from the provisioning apparatus;

FIG. 10 shows a schematic depiction of the handling of the second end of the catheter upon removal from the provisioning apparatus;

FIG. 11 shows a schematic depiction of the approach (second option) in removing the rolled up catheter as a whole from the provisioning apparatus;

FIG. 12 shows a schematic instruction for handling the removed, rolled up catheter after removal from the provisioning apparatus;

FIG. 13 shows a schematic depiction of a third option for removing the catheter from the provisioning apparatus; and

FIG. 14 shows a schematic depiction of a further step of the third option for removing the catheter from the provisioning apparatus.

### DETAILED DESCRIPTION

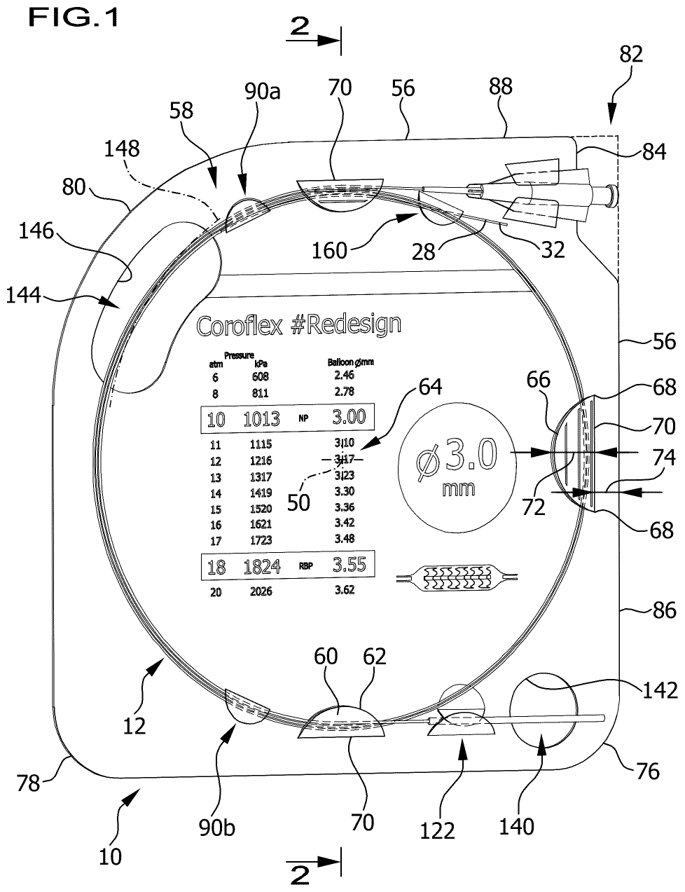

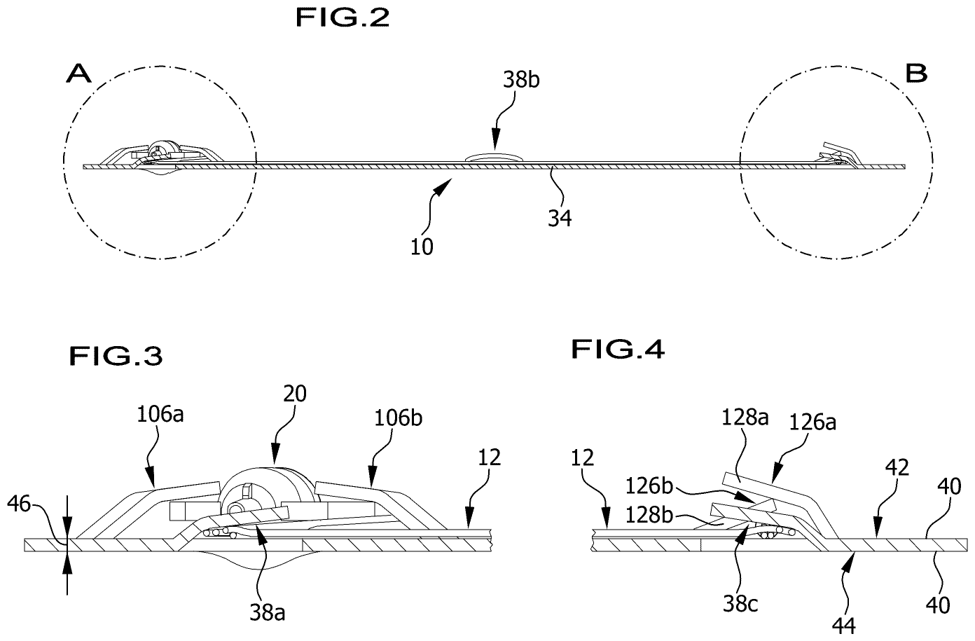

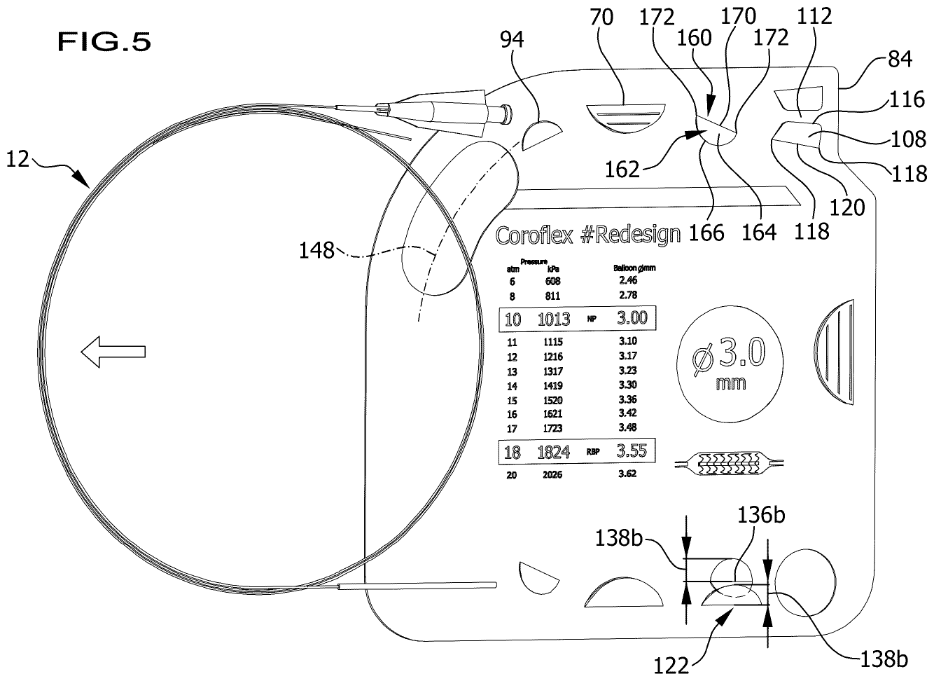

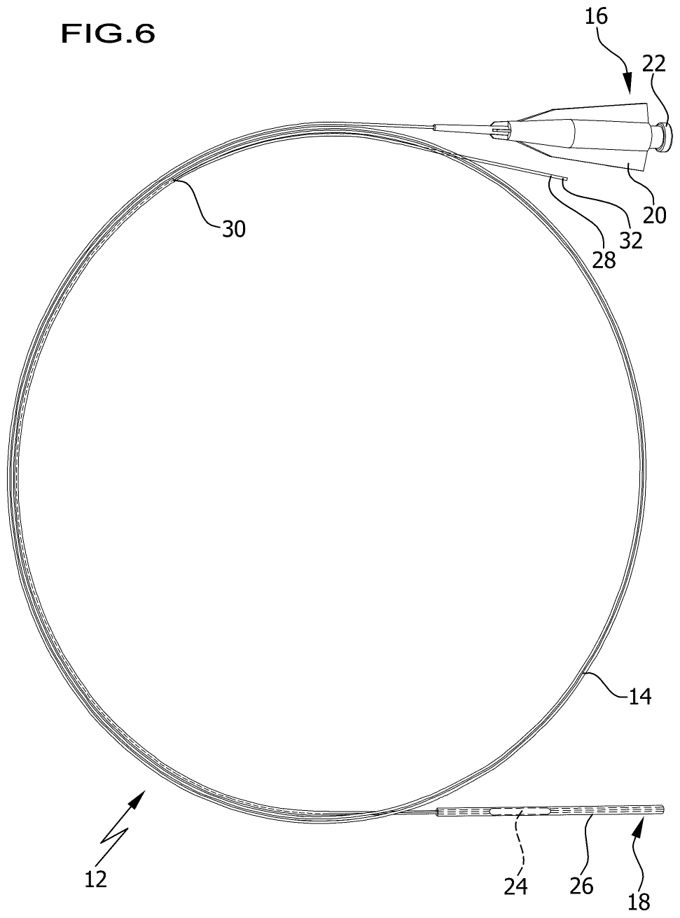

Schematically depicted in FIG. 1 is a first embodiment of a provisioning apparatus, denoted as a whole with the reference numeral 10, for the storage and transport of a catheter 12. The catheter 12 is configured in the form of a balloon catheter 14 for percutaneous transluminal coronary angioplasty.

The catheter 12 has a first end 16 and a second end 18. Arranged or formed on the first end 16 is a connection adapter 20 with a so-called luer lock connector 22. Arranged or formed in the region of the second end 18 is an inflatable balloon 24. Commencing from the second end 18, a protective cover 26 is slid over that surrounds the balloon 24 in a protective manner. The catheter 12 comprises a fluid channel, which is not depicted in the Figures, that fluidically connects the connection adapter 20 and the balloon 24.

Furthermore, a wire channel for a wire 28 is formed on the catheter 12. The wire channel, which is not depicted in the Figures, extends commencing from the second end 18 in the proximal direction up to an insertion opening 30, which is arranged or formed on the catheter 12 between the first end 16 and the second end 18. A distance of the insertion opening 30 from the second end is smaller than a distance from the first end 16.

A wire 28 is positioned in the wire channel of the catheter 12. Said wire is longer than the wire channel, such that a proximal end 32 pointing in the direction toward the connection adapter 20 projects out of the insertion opening 30. The wire 28 is configured in the form of a transport wire, which is removed before a medical procedure.

The provisioning apparatus 10 comprises a support 34 on which a holding device 36 is arranged or formed for holding the catheter 12 rolled up in an annular manner in a storage position. The holding device 36 comprises at least three holding elements 38a, 38b, and 38c, namely exactly three in the case of the embodiment depicted in the Figures, which in the storage position are in engagement with the catheter 12.

The three holding elements 38a, 38b, and 38c are, as can be easily seen in the Figures, arranged or formed spatially separate from one another on the support 34. However, they cooperate to hold the catheter 12 on the support 34.

The provisioning apparatus 10, namely the support 34, is made of a flat material, which forms a support blank. In particular cardboard and paperboard sheets are suitable as flat material.

In an alternative embodiment, the flat material for forming the support 34 is made of plastic.

The support 34 is optionally provided with a liquid-repellent coating 40, namely both on a front side 42 and on a rear side 44. The coating 40 is made of a plastic material. In particular, the coating may be configured in the form of a plastic film.

The plastic material of which the flat material is made or with which it is coated is or contains polypropylene (PP), high-density polyethylene (HDPE), polylactic acid (PLA, in particular PLLA), polyamide (PA), polyethylene (PE), low-density polyethylene (LDPE), and/or polyethylene terephthalate (PET).

The provisioning apparatus 10 as a whole is formed in one piece, namely monolithically.

The support 34 has a support thickness 46 in a range of about 0.05 mm to about 1 mm. In particular, the support thickness 46 may have a value in a range of about 0.2 mm to about 0.4 mm.

The holding device 36 is configured to hold the catheter 12 on the front side 42 of the support 34, as will be described in detail in the following.

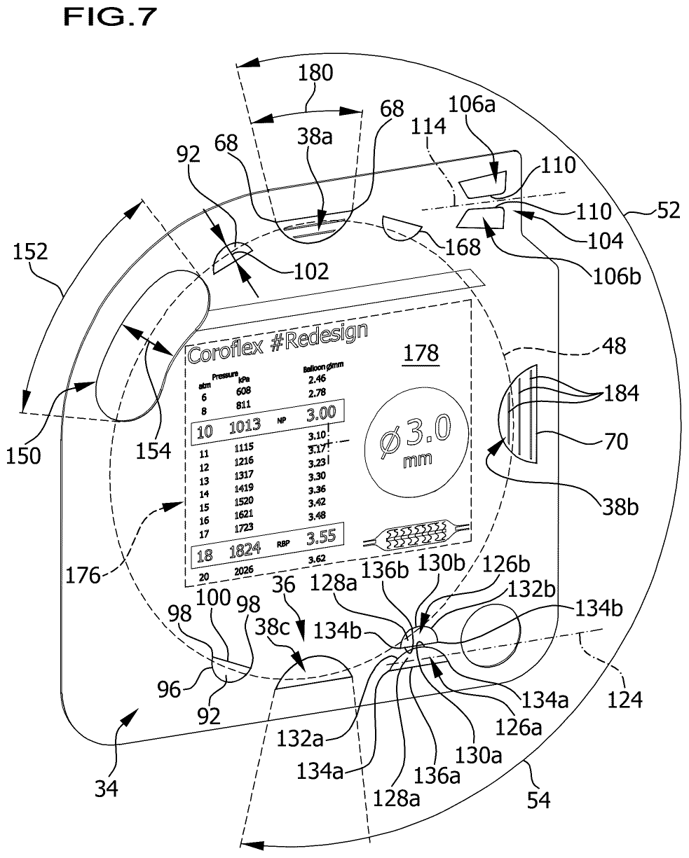

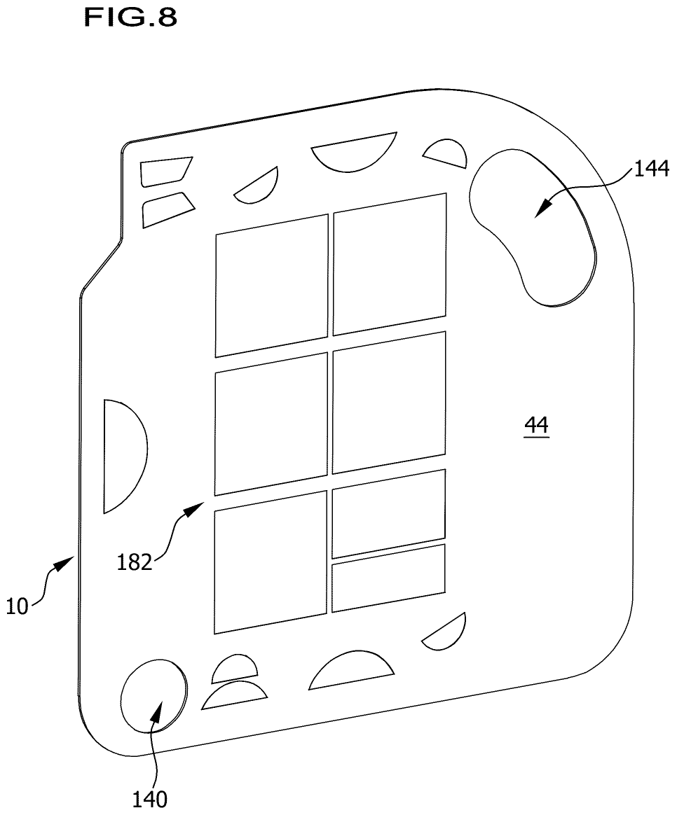

The three holding elements 38a, 38b, 38c are positioned along a holding circle 48, which is drawn schematically in FIG. 7 with dashed lines. The holding circle 48 defines a holding circle midpoint 50.

Two of the three holding elements 38a, 38b, and 38c, namely the holding elements 38a and 38c, are positioned point-symmetrically on the support 34 relative to the holding circle midpoint 50.

The three holding elements 38a, 38b, and 38c define a holding region 52, which extends around the holding circle midpoint 50. The holding region 52 of the holding circle 48 defines a holding circle circumferential angle 54 of at least 180°. Preferably, a value of the holding circle circumferential angle 54 is in a range between 180° and 240°. In the embodiment depicted in the Figures, the holding circle circumferential angle 54 is nearly 200°.

The support 34 has an outer peripheral support rim 56. The entire area of the front side 42 surrounded by the support rim 56 encloses a storage region 58. The catheter 12 in the storage position is arranged within the storage region 58, with one exception which will be discussed later.

The three holding elements 38a, 38b, and 38c are configured in the form of holding tabs 60. A free holding tab end 62 of the holding tab 60 is oriented pointing in the direction toward a support center 64 of the support 34. In the embodiment depicted in the Figures, the holding circle midpoint 50 forms the support center 64.

The holding tab 60 is formed by a holding cut line 66 formed on the support 34. Said holding cut line has two free ends 68. A holding tab base line 70 of the holding tab 60 connects the two free ends 68.

A free holding tab length 72 of the holding tab 60 is defined by a distance of the free holding tab end 62 from the holding tab base line 70. The holding cut line 66 is convexly curved pointing in the direction toward the support center 64. It has the form of a circular arc section.

The holding tab base lines 70 of the two holding tabs 60, which form the holding elements 38a and 38c, extend in parallel with one another.

The holding tab base lines 70 of the holding tabs 60, which form the holding elements 38a and 38b, extend transversely, namely perpendicularly to one another. The holding tab base lines 70 of the holding tabs 60, which form the holding elements 38b and 38c, also extend transversely, namely perpendicularly to one another.

A distance 74 of the holding tab base line 70 of the holding tab 60, which forms the holding element 38b, from the support rim 56 is smaller than its free holding tab length 72.

The three holding tabs 60 forming the holding elements 38a, 38b, and 38c are each positioned near the support rim 56. The respective holding tab base lines 70 extend in parallel or substantially in parallel with the support rim 56.

The support 34 has a substantially rectangular shape. Two adjacent corners 76 and 78 are rounded with a small radius, while a further corner 80 adjacent to the corner 78 is rounded with a large radius. In the region of a fourth corner 82, which is defined between the corners 76 and 80, an adapter cutout 84 is formed, which shortens both rectilinearly extending support rim portions 86 and 88 of the support rim 56 that define the corner 82.

The holding device 36 also comprises two retaining elements 90a and 90b. They are configured to prevent a movement of the catheter 12 in the storage position in the direction toward the holding circle midpoint 50.

The retaining elements 90a, and 90b are configured in the form of retaining tabs 92. A free retaining tab end 94 of the retaining tab 92 is oriented pointing in the radial direction away from the support center 64.

The retaining tab 62 is formed by a retaining cut line 96 on the support 34. The retaining cut line 96 has two free ends 98. They are connected by a retaining tab base line 100. A free retaining tab length 102 of the retaining tab 92 is defined by a distance of the retaining tab end 94 from the retaining tab base line 100. The retaining cut line 96 is concavely curved pointing in the direction toward the support center 64, namely in the form of a circular arc section.

The two retaining elements 90a and 90b are positioned along the holding circle 48, namely between the two holding elements 38a and 38c that have the greatest angular distance from one another. The angular distance between the holding elements 38a and 38b is roughly as large as the angular distance between the holding elements 38b and 38c and is about 80° in each case. By contrast, the angular distance between the holding elements 38a and 38c is about 170°.

The provisioning apparatus 10 further comprises an adapter holding device 104 for holding the connection adapter 20 arranged on the first end 16 of the catheter 12. The adapter holding device 104 comprises two adapter holding elements 106a and 106b.

The two cooperating adapter holding elements 106a and 106b are configured in the form of adapter holding tabs 108. Free adapter tab ends 110 of the two adapter holding elements 106a and 106b are oriented pointing toward one another.

The adapter tab ends 110 pointing toward one another are spaced at a distance from one another and thus form an adapter holding web 112 on the support 34.

The adapter holding device 104 defines an adapter holding longitudinal axis 114. The adapter holding web 112 extends in parallel with the adapter holding longitudinal axis 114.

The adapter holding device 104 is configured symmetrically relative to the adapter holding longitudinal axis 114. The adapter holding tab 108 is defined by an adapter cut line 116 comprising three rectilinear portions, such that a substantially quadrangular adapter holding tab 108 is formed. Free ends 118 of the adapter cut line 116 are connected to one another by way of an adapter holding tab base line 120. The two adapter holding tab base lines 120 of the two adapter holding tabs 108 extend in the direction toward the adapter cutout inclined slightly away from one another and thus not in parallel with the adapter holding longitudinal axis 114. Alternatively and also preferably, the two adapter holding tab base lines 120 extend in parallel with one another, in particular in parallel with the support rim portion 88.

The holding tab base line 70 of the holding tab 60, which forms the holding element 38a, extends in parallel with the adapter holding longitudinal axis 114.

As already explained, the adapter cutout 84 is formed adjacent to the adapter holding device 104 commencing from the support rim 56. By provisioning the adapter cutout 84, a portion of the support 34 is missing that would be delimited in the direction toward the corner 82 by extensions of the support rim portions 86 and 88.

The provisioning apparatus 10 further comprises a balloon holding device 122 for the balloon 24, which is arranged or formed in the region of the second end 18 of the catheter 12. The balloon holding device 122 defines a balloon holding longitudinal axis 124. The balloon holding device 122 is configured asymmetrically relative to the balloon holding longitudinal axis 124. It comprises two balloon holding elements 126a and 126b.

The two cooperating balloon holding elements 126a and 126b are configured in the form of balloon holding tabs 128a and 128b. Free balloon holding tab ends 130a and 130b of the balloon holding tabs 128a and 128b point in the same direction in an undeformed starting position of the provisioning apparatus 10, i.e., when only the cuts to form them have been applied to the support. This is schematically depicted in FIG. 7. The free balloon holding tab ends 130a and 130b point in the direction of the support rim portion 88.

The balloon holding tabs 128a and 128b are formed by balloon holding cut lines 132a and 132b on the support 34. Free ends 134a and 134b of the balloon holding cut lines 132a and 132b are connected to one another by a balloon holding tab base line 136a and 136b, respectively. A free balloon holding tab length 138a and 138b is defined by a distance of the respective free balloon holding tab end 130a and 130b from the respective balloon holding tab base line 136a and 136b.

The balloon holding cut lines 132a and 132b are of curved configuration, namely in the form of circular arc sections.

The two balloon holding tab base lines 136a and 136b extend in parallel with one another and in parallel with the balloon holding longitudinal axis 124.

The balloon holding cut lines 132a and 132b of the two cooperating balloon holding tabs 128a and 128b differ in length and also have different curvature radii. The balloon holding cut line 132a is longer than the balloon holding cut line 132b. By contrast, the balloon holding cut line 132b is more curved than the balloon holding cut line 132a. Thus, a curvature radius of the balloon holding cut line 132b is smaller than a curvature radius of the balloon holding cut line 132a. Still, the two balloon holding tab lengths 138a and 138b of the two balloon holding tabs 128a and 128b are substantially equal.

Furthermore, the holding tab base line 70 of the holding tab 60, which forms the holding elements 38c, is oriented in parallel with the balloon holding longitudinal axis 124. Thus, the second end of the catheter 12 can be guided tangentially away from the wound up region to the balloon holding device 122.

The provisioning apparatus 10 further comprises a tip recess 140. It is configured in the form of a perforation 142 on the support 34. The tip recess 140 is positioned adjacent to the balloon holding device 122, namely in the region of the corner 76, pointing in the direction of the balloon holding longitudinal axis 124. The balloon holding longitudinal axis 124 points in the direction of the support rim portion 86.

As shown schematically in FIG. 1, the second end 18 of the catheter 12 is positioned in the region of the tip recess 140 in the storage position.

The tip recess 140 is further dimensioned in such a way that a user is able to grasp the second end 18 of the catheter 12 held on the support 34 with two fingers.

Furthermore, a removal recess 144 is arranged or formed on the support 34. In the storage position, as schematically depicted in FIG. 1, at least two catheter portions or windings of the catheter 12 held rolled up on the support 34, namely three portions in the case of the embodiment depicted, extend in the region of the removal recess 144.

The removal recess 144 is configured in the form of a perforation 146 on the support 34. It is shaped in the form of an elongate hole, namely in the form of a circular ring section 150.

A center line 148 of the circular ring section 150 has a curvature that corresponds roughly to a curvature of the catheter 12 held rolled up on the provisioning apparatus 10 in the storage position, as is schematically depicted in FIG. 1.

The circular ring section 150 extends over an angular range 152 of about 30° to about 60°. In particular, the angular range may be about 40° to about 50°. In the embodiment depicted in the Figures, the angular range extends over about 45°.

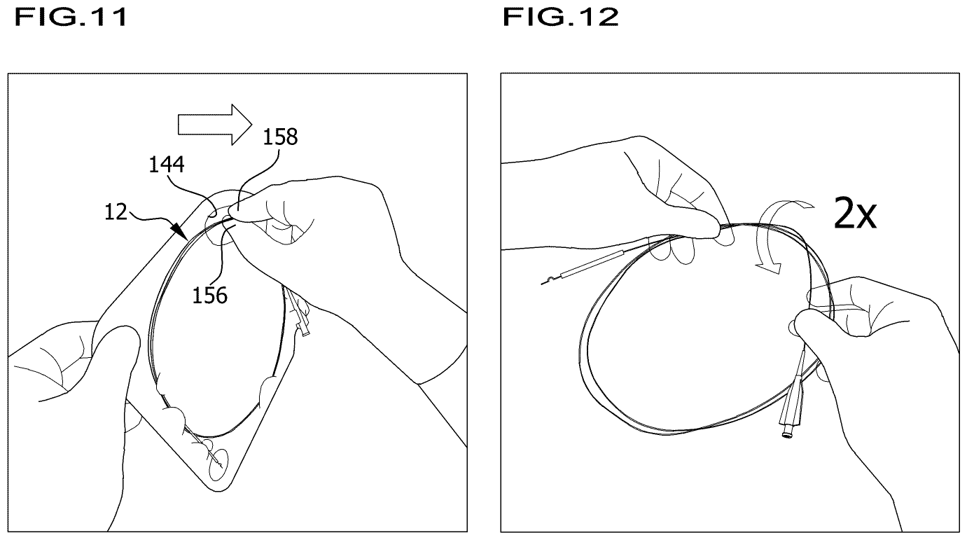

The removal recess 144 has a width 154 in the radial direction relative to the holding circle midpoint 50. The width 154 is dimensioned such that it is possible to grasp the catheter 12 held flat on the support 34 with two fingers, namely a thumb 156 and an index finger 158, as schematically depicted in FIG. 11.

The removal recess 144 is further positioned on the holding circle 48, namely between the two holding elements 38a and 38c that have the greatest angular distance from one another, as already discussed in detail above. The circular ring section 150 may comprise a portion of the holding circle 48.

The provisioning apparatus 10 further comprises a wire holding device 160 for holding the end 32 of the wire 28 projecting out of the wire channel. In the embodiment depicted in the Figures, it is positioned in the region between the holding element 38a and the adapter holding device 104.

The wire holding device 160 comprises a wire holding element 162. The wire holding element 162 is configured in the form of a wire holding tab 164. A free wire holding tab end 166 of the wire holding tab 164 is oriented pointing in the direction toward the support center 64.

To form the wire holding tab 164, a wire holding cut line 168 is formed on the support 34. The wire holding tab 164 defines a wire holding tab base line 170. Said base line connects free ends 172 of the wire holding cut line 168 to one another. A wire holding tab length 174 of the wire holding tab 164 is defined by a distance of the wire holding tab end 166 from the wire holding tab base line 170.

The provisioning apparatus 10 further comprises a data storage element 176 for storing characteristic data about the catheter 12 held on the provisioning apparatus 10. Characteristic data about the catheter are, in particular, its length, a catheter sheath diameter, an inflation pressure, and a balloon diameter, the latter being dependent on the inflation pressure.

The data storage element 176 is configured in the form of a printed data region 178, namely on the front side 42 of the support 34. The data storage element 176 is optically readable. The characteristic data are printed in Latin characters, so that they are able to be directly read by a user.

The holding elements 38a, 38b, and 38c extend over a circumferential angle 180 of about 10°. The circumferential angle is defined by the opening angle commencing from the holding circle midpoint 50 to the free ends 68 of the holding cut lines 66. In this way, the holding elements 38a, 38b, and 38c can each hold the rolled up catheter 12 over a circumferential angle 180 of about 10°.

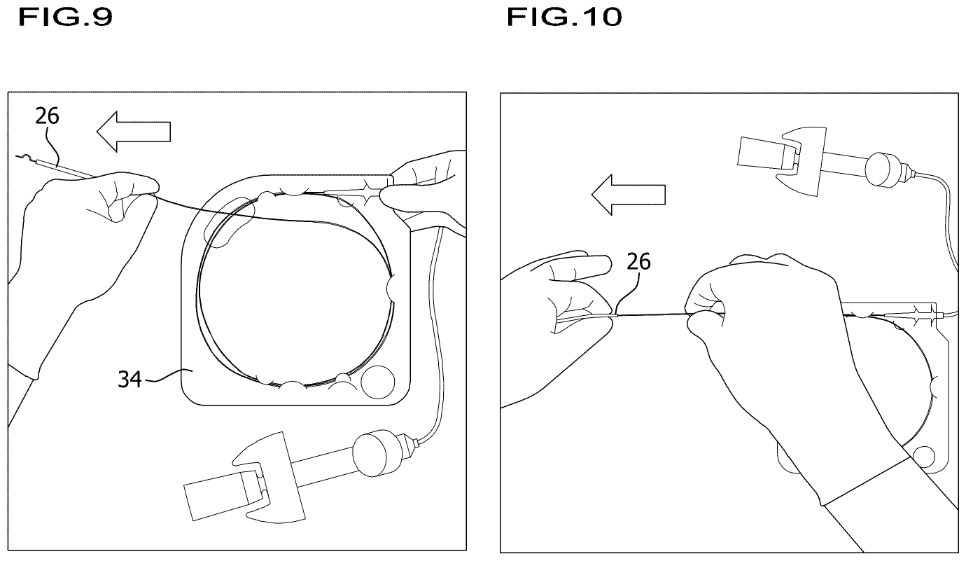

Operating instructions 182 for using and handling the provisioning apparatus 10 and for handling the catheter 12 are printed on the rear side 44. Said instructions may be the depictions in FIGS. 9 to 14, which will be briefly explained in the following. The operating instructions 182 also form a data region and thus define a data storage element.

The particular design of the provisioning apparatus 10 enables three different removal options of the catheter 12 from the support 34. FIG. 9 shows a first variant. A user hereby grasps the support 34 with their right hand in the region of the adapter holding device 104 and thus securely holds the connection adapter 20. The user can now grasp the second end 18 of the catheter 12 with their other hand and remove the catheter 12 from the support 34 winding by winding. In particular, the user can leave the catheter 12 on the support 34 such that only the portion of the catheter 12 that comprises the wire channel is removed from the support 34. FIG. 10 shows how the connection adapter 20 is still held on the adapter holding device 104, but is already coupled to a flushing device. Furthermore, the protective cover 26 can be removed from the second end 18 in a simple manner when the second end 18 has been released from the support 34.

According to a second variant, the provisioning apparatus 10 also makes it possible to bring the rolled up catheter 12 and the three holding elements 38a, 38b, and 38c out of engagement without bringing the catheter 12 into an elongated form. To achieve this, a user must only grasp the catheter 12 in the region of the removal recess 144 with two fingers, the thumb 156 and index finger 158 being shown in FIG. 11 as an example. If the user holds the support 34 in the region of the corner 78 with their other hand, they can remove the rolled up catheter 12 as a whole. In order to prevent the catheter 12 from unrolling, the user should now, as schematically depicted in FIG. 12, wrap the first end 16 with the connection adapter 20 around the rolled up catheter 12 twice.

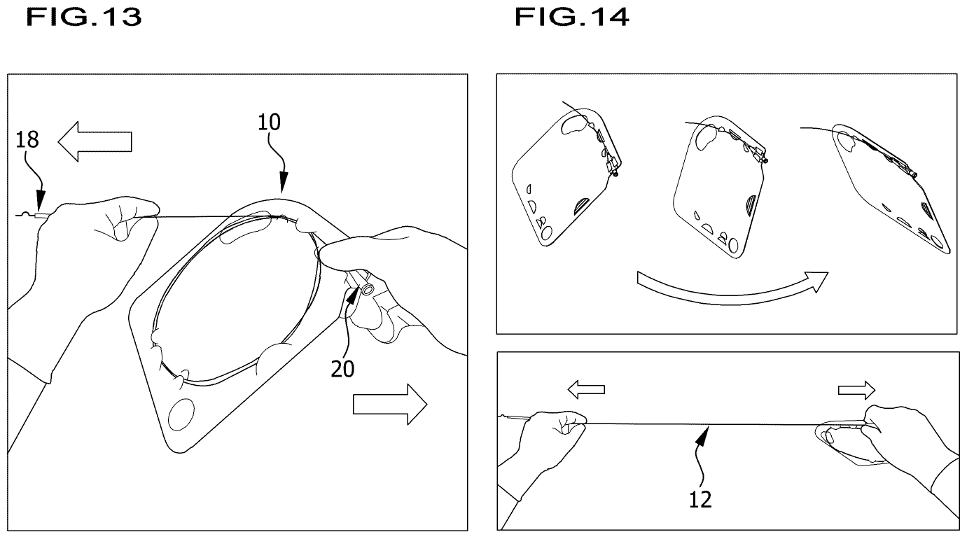

A third option for removing the catheter 12 from the provisioning apparatus 10 is schematically depicted in FIGS. 13 and 14. A user hereby grasps the second end 18 by reaching into the tip recess 140 with one hand. They hold the connection adapter 20 securely with the support 34 with the other hand. When they pull the second end 18 to the left in the direction of the arrow, the catheter 12 unrolls and the user can hold the catheter 12 elongated rectilinearly between both hands.

In the embodiment described in the Figures, the catheter 12 in the storage position is held on the provisioning apparatus 10 in such a way that the first end 16 and the second end 18 are oriented transversely, namely perpendicularly, to the support rim 56, namely to the support rim portion 86.

The three holding elements 38a, 38b, and 38c may optionally each comprise a plurality of fold lines 184, which extend in parallel with one another and in parallel with the holding tab base line 70. This makes it possible to fold or bend the holding tabs 60 along the fold lines such that they extend in the shape of an arc and engage over the rolled up catheter 12 in an arcuate manner.

The two balloon holding tabs 128a and 128b are used as follows to hold the catheter 12 in the region of the balloon 24. The balloon holding tab 128b is flipped around the balloon holding tab base line 136b in the direction toward the balloon holding tab 128a, but without folding or bending it relative to the support 34 along the balloon holding tab base line 136b. It is slid with the free balloon holding tab end 130b under the balloon holding tab 128a. Due to the somewhat elastic property of the material from which the support 34 is made, the balloon holding tab 128a is pushed somewhat away from the front side 42, such that the balloon holding tabs 128a and 128b together wrap arcuately over the balloon 24, as is schematically depicted in FIG. 4.

The described provisioning apparatus makes it possible to store the catheter 12 flat on the front side 42 of the support 34. In particular, it is thus possible to store the catheter 12 as flat as possible overall.

Furthermore, the provisioning apparatus enables three variants of removal as described above.

When the second end is removed from the support 34, the protective cover 26 and the wire 28 can be removed. The catheter 12 can be threaded onto the actual guide wire used for the medical procedure and can then be inserted into a guide catheter.

A further advantage of the described provisioning apparatus 10 is that the catheter 12 remains on the support 34 until the final removal of the catheter 12, in particular until removal of the connection adapter 20 from the adapter holding device 104, and a user always has all information regarding the catheter 12 used directly available to them, because the data region 178 continues to be coupled to the catheter 12 by way of the connection adapter 20.

### Reference numeral list

10 provisioning apparatus
12 catheter
14 balloon catheter
16 first end
18 second end
20 connection adapter
22 luer lock connector
24 balloon
26 protective cover
28 wire
30 insertion opening
32 end
34 support
36 holding device
38a, 38b, 38c holding element
40 coating
42 front side
44 rear side
46 support thickness
48 holding circle
50 holding circle midpoint
52 holding region
54 holding circle circumferential angle
56 support rim
58 storage region
60 holding tab
62 holding tab end
64 support center
66 holding cut line
68 end
70 holding tab base line
72 holding tab length
74 distance
76 corner
78 corner
80 corner
82 corner
84 adapter cutout
86 support rim portion
88 support rim portion
90a, 90b retaining element
92 retaining tab
94 retaining tab end
96 retaining cut line
98 end
100 retaining tab base line
102 retaining tab length
104 adapter holding device
106a, 106b adapter holding element
108 adapter holding tab
110 adapter tab end
112 adapter holding web
114 adapter holding longitudinal axis
116 adapter cut line
118 end
120 adapter holding tab base line
122 balloon holding device
124 balloon holding longitudinal axis
126a, 126b balloon holding element
128a, 128b balloon holding tab
130a, 130b balloon holding tab end
132a, 132b balloon holding cut line
134a, 134b end
136a, 136b balloon holding tab base line
138a, 138b balloon holding tab length
140 tip recess
142 perforation
144 removal recess
146 perforation
148 center line
150 circular ring section
152 angular range
154 width
156 thumb
158 index finger
160 wire holding device
162 wire holding element
164 wire holding tab
166 wire holding tab end
168 wire holding cut line
170 wire holding tab base line
172 end
174 wire holding tab length
176 data storage element
178 data region
180 circumferential angle
182 operating instruction
184 fold line

1. A provisioning apparatus for storage and transport of a catheter, the provisioning apparatus comprising:
a support; and
a holding device,
the holding device arranged or formed on the support for holding the catheter when the catheter is rolled up in an annular manner in a rolled up storage position,
the holding device comprising at least three holding elements that are in engagement with the catheter in the rolled up storage position,
the at least three holding elements arranged or formed spatially separate from one another on the support,
the at least three holding elements positioned along a holding circle that defines a holding circle midpoint,
at least one of the at least three holding elements comprising a holding tab having a free holding tab end,
the free holding tab end pointing toward a support center of the support, and
the holding device further comprising at least two retaining elements for preventing movement of the catheter in the rolled up storage position toward the holding circle midpoint.

2. The provisioning apparatus according to claim 1, wherein, when the catheter is in the rolled up storage position, the provisioning apparatus is configured to disengage the catheter from the at least three holding elements without bringing the catheter into an elongated form.

3. The provisioning apparatus according to claim 1, wherein two of the at least three holding elements are positioned point-symmetrically relative to the holding circle midpoint.

4. The provisioning apparatus according to claim 3, wherein the at least three holding elements define a holding region corresponding to a holding circle circumferential angle of the holding circle of at least 180°.

5. The provisioning apparatus according to claim 4, wherein the holding circle circumferential angle is in a range between 180° and 240°.

6. The provisioning apparatus according to claim 1, wherein the holding tab is formed by a holding cut line formed on the support and having two free ends, wherein the holding tab defines a holding tab base line, which connects the two free ends of the holding cut line, and wherein a free holding tab length of the holding tab is defined by a distance of the free holding tab end from the holding tab base line.

7. The provisioning apparatus according to claim 1, wherein at least one, in particular all, of the at least two retaining elements is/are configured as a retaining tab, and wherein a free retaining tab end of the retaining tab is oriented pointing in a radial direction away from the support center.

8. The provisioning apparatus according to claim 1, further comprising an adapter holding device for a connection adapter arranged or formed on a first end of the catheter.

9. The provisioning apparatus according to claim 8, wherein the adapter holding device comprises at least two adapter holding elements.

10. The provisioning apparatus according to claim 9, wherein:
the at least two adapter holding elements comprise two cooperating adapter holding elements configured as adapter holding tabs,
the two cooperating adapter holding elements comprise free adapter tab ends, and
the free adapter tab ends point toward one another.

11. The provisioning apparatus according to claim 1, further comprising a balloon holding device for an inflatable balloon arranged or formed in a region of a second end of the catheter.

12. The provisioning apparatus according to claim 11, wherein:
the balloon holding device comprises at least two balloon holding elements,
the at least two balloon holding elements comprise two cooperating balloon holding elements configured as balloon holding tabs,
the balloon holding tabs comprising free balloon holding tab ends, and
the free balloon holding tab ends point in a common direction when the provisioning apparatus is in an undeformed starting position.

13. The provisioning apparatus according to claim 1, wherein:
a tip recess is arranged or formed on the support,
a second end of the catheter in the rolled up storage position is positioned in a region of the tip recess.

14. The provisioning apparatus according to claim 1, wherein:
a removal recess is arranged or formed on the support, and
when the catheter is in the rolled up storage position, at least two catheter portions of the catheter are positioned in a region of the removal recess.

15. The provisioning apparatus according to claim 1, further comprising a wire holding device for holding a free end of a wire that projects from of a wire channel of the catheter out of an opening of the wire channel pointing toward a first end of the catheter.

16. The provisioning apparatus according to claim 15, wherein:
the wire holding device comprises at least one wire holding element, the at least one wire holding element is configured as a wire holding tab, and the wire holding tab has a free wire holding tab end pointing toward the support center.

17. The provisioning apparatus according to claim 1, wherein the provisioning apparatus is made of a flat material.

18. The provisioning apparatus according to claim 1, wherein the provisioning apparatus is formed in a one piece homogeous body of unitary construction.

19. The provisioning apparatus according to claim 1, further comprising at least one data storage element for storing characteristic data about the catheter.

20. The provisioning apparatus according to claim 1, further comprising the catheter.
